(19) United States
(12) Patent Application Publication
(10) Pub. No.: US 2023/0381481 A1
(43) Pub. Date: Nov. 30, 2023

(54) METHODS AND SYSTEMS FOR PERCUTANEOUS ACCESS

(71) Applicant: Steryl, Inc., Atherton, CA (US)

(72) Inventor: Michael J. Pizzato, Atherton, CA (US)

(73) Assignee: Steryl, Inc., Atherton, CA (US)

(21) Appl. No.: 18/359,776

(22) Filed: Jul. 26, 2023

Related U.S. Application Data
(63) Continuation-in-part of application No. PCT/US22/14503, filed on Jan. 31, 2022.
(60) Provisional application No. 63/143,224, filed on Jan. 29, 2021.

(51) Int. Cl. A61M 39/02

(52) U.S. Cl. CPC A61M 39/0247; A61M 2039/0258; A61M 2039/0282; A61M 2039/0294

(57) ABSTRACT

A percutaneous access system provides placement of an access catheter through a tissue access tract into a patient's vasculature. The system includes an access needle having a narrow width, a dilator or carrier dilator having a tapered distal end and an expanded-width proximal portion, and a radially expandable sheath having a lumen configured to receive the needle when said sheath in a radially constricted configuration. The sheath is radially expanded by advancement of the dilator or dilator carrier through the sheath lumen to allow advancement of the access catheter therethrough. In some examples, the dilator is removed prior to introducing the catheter into the expanded sheath. In other examples, the catheter is introduced in the carrier dilator that radially expands the sheath.

### CROSS-REFERENCE TO RELATED APPLICATION

The application is a continuation-in-part of PCT Application No. PCT/US22/14503 (Attorney Docket No. 60346-703601), filed Jan. 31, 2022, which claims the benefit of U.S. Provisional Application No. 63/143,224 (Attorney Docket No. 62346-703.101), filed on Jan. 29, 2021, the full disclosure of which is incorporated herein by reference.

### BACKGROUND OF THE INVENTION

### 1. Field of the Invention

The present invention relates generally to medical devices and methods. More particularly, the present invention and methods relate to an expandable radially expandable sheath device used for central venous catheter (CVC) and other percutaneous access.

Introducing a device into the body requires technical skill and risks a wide range of vascular complications including infection. The successful insertion of a device relies on the technique used and the skillset of the operator. The Seldinger technique and various modifications of the Seldinger technique have been utilized since 1953 and require more than a handful of steps. Some of these steps require practice and dexterity to master such as holding the needle tip steady within the vessel to maintain position. This can be a balancing act as it must be done at the same time as removing the syringe, reaching for the wire, and inserting the wire. If position within the vessel is not maintained, the operator will be unsuccessful in advancing the wire and/or cause additional trauma (puncturing the vessel backwall, surrounding arteries/vessels, lung, etc.).

A modification of the Seldinger technique using a catheter over the needle (CON) approach helps reduce the skill and dexterity needed by an operator by allowing them to advance a small angio-catheter off the needle into the vessel as soon as they receive confirmation of flashback, increasing the success of access. However, this technique is not always used as it adds additional steps to the procedure.

Additionally, operators find that handling the guidewire can be cumbersome due to its floppiness and need to always hold the guidewire to maintain position. Guidewires have been reported to become entrapped within the body due to not securing them during insertion. Threading devices such as the dilator and catheter over the guidewire requires dexterity and good eyesight and to quickly align the small opening in the tip of the device and slide over the guidewire.

Studies have shown that operators with more experience have a higher success rate of first attempts and a lower incidence of complications. Creating a new device and method to place a vascular device that reduces the skill and dexterity needed while reducing the number of steps involved may lead to a higher number of successful first time attempts and a lower complication rate across all operators.

Clinicians and hospital administrators are committed to reducing device-associated infections, provide better patient outcomes, and reduce healthcare expenses to the system. Both catheter related blood stream infections (CRBSI) and central line associated blood stream infections (CLABSI) are a concern due to the high degree of patient morbidity and increased healthcare costs. Annually, it is estimated that 80,000 device related blood stream infections in the ICU, resulting in $296 million to $2.3 billion in additional costs. Mortality rate is 4% to 20% and results in approximately 2,400 to 20,000 deaths.

Vascular access is treated as a sterile procedure, however even with proper skin preparation with the most effective antiseptic solutions and methods, complete sterility can never be achieved due to the bacteria that resides in the crevasses, sebaceous glands, and deeper layers of skin. Contamination of the skin is unavoidable despite the best efforts of the clinical team. Over 60% of catheter related infections are due to extraluminal contamination caused by skin organisms, the majority coming directly from the patient's skin. Furthermore, research has found that 80% of the bacteria genera found on a vascular catheter directly matched a patient's skin swab at the catheter insertion site. Even smaller devices, such as needles, can carry bacteria into the tissue tract and Wang et al. demonstrated that transfer of bacteria increased with needle diameter for 30G, 25G and 18G needles. Given these facts and the mortality rates of both CRBSI and CLABSI, there has been a major focus to reduce CLABSIs and new technology is needed to reduce complications and enable safer vascular access.

To establish vascular access, the Seldinger technique, first described in 1953, is the standard of care and still used to this day. This technique utilizes a needle, guidewire, scalpel, dilator, and sheath to gain access to the target vessel. Using this basic technique, a needle is used to access the vasculature. A guidewire is passed through the needle into the blood vessel and the needle is withdrawn over the guidewire, leaving the guidewire as the pathway to the vessel. Next, a scalpel is used to nick the skin at the guidewire exit to make room for the dilator. A dilator is passed over the guidewire to enlarge the diameter of the tissue tract and removed. Finally, the catheter is advanced over the guidewire to its final resting position and the guidewire is removed. This procedure is summarized in FIG. 1.

With the Seldinger technique, multiple instruments are being introduced through the skin. Each time an instrument moves in and out of the tissue tract, the risk of carrying bacteria from the skin into the tissue tract and seeding the catheter increases. The skin insertion site is often contaminated and frequently results in the contamination of the of the catheter distal tip, needle, guidewire, scalpel, and dilator in many cases within 90 minutes. Bacterial seeding of the tissue tract and catheter upon insertion increases the chances that biofilm will develop and potentially lead to infection.

With the Seldinger technique, there are multiple steps for potential skin flora contamination during catheter insertion. Contamination may happen during needle insertion, guidewire placement, nicking the skin with the scalpel, dilating the tract, and inserting the catheter and may result in contamination of the dwelling device on both the inner and out lumen surface. Given the many “skin to device” interactions with the Seldinger technique, each time increasing the risk of contamination, it would be desirable to utilize an improved device and method that minimizes the number of skin-instrument interactions and reduces the number of steps required to gain percutaneous access. See FIG. 2.

Many current devices have focused on inhibiting the bacteria after it attaches to the catheter by impregnating chlorohexidine, antibiotics, or silver particles on the catheter instead of preventing bacteria seeding the tissue tract and catheter during insertion. New devices and methods to reduce bacteria entering the access tract upon placement and contaminating indwelling devices are urgently needed to enable a safer and more cost-effective percutaneous access, a value proposition benefiting patients, providers, hospitals, and payers alike. It would be further desirable to provide catheter access devices and methods capable of reducing both the complexity and duration of catheter access protocols, particularly by reducing the number of steps needed for percutaneous catheter introduction. At least some of these objectives will be met by the inventions described herein.

### 2. Listing of Background Art

Relevant patents and publications include: US2020/009402; US2013/0030369; U.S. Pat. Nos. 10,850,071; 10,682,157; 9,884,169; 9,415,186; 8,597,277; 7,144,386; 5,183,464; 4,306,562 (RE31855); U.S. Pat. No. 3,570,485; WO2005/086716; WO2006/084154; and WO2007/047905.

### BRIEF SUMMARY OF THE INVENTION

In a first aspect, the present invention provides a percutaneous access system for placement of a venous or other access catheter through a tissue access tract into a patient's vasculature. The percutaneous access system comprises an access needle having a narrow width, typically having a diameter from 0.5 mm to 1.5 mm, typically a 21 Ga (0.83 mm OD) to 18 Ga (1.27 mm OD) needle; a dilator having a tapered distal end and an expanded-width proximal portion, typically having a diameter from 3French (F) (1 mm) to 15F (5 mm); and a radially expandable sheath having a lumen configured to receive the needle when said sheath in a radially constricted configuration. Usually, the lumen will have an inner diameter which is no more than 1 mm greater that the needle when sheath is in its radially constricted configuration. The percutaneous access sheath is sufficiently malleable to be reshaped from the radially constricted configuration to a radially expanded configuration without breaking or cracking by advancement of the dilator through the sheath lumen and sufficiently stiff to remain radially expanded in the tissue access tract after the dilator has been removed. The percutaneous access sheath is preferably further configured to be axially split after radial expansion. The width of the sheath lumen after radial expansion will usually match that of the outer diameter of the dilator, sometimes being slightly larger due to pivoting or other unsteady motion of the dilator as it is introduced and/or removed from the sheath or sometimes being slightly smaller as a result of a small recoil of the sheath after removal of the dilator. The exact width of the sheath lumen after radial expansion is not critical and need only be sufficient to receive the subsequent passage of the access catheter.

In some embodiments, the percutaneous access systems of the present invention may be part of a kit or package which further comprises the access catheter that is configured to be introduced through the radially expanded lumen of the radially expandable sheath. Those embodiments which further comprise the access catheter often also comprise a guidewire configured to be received in a guidewire lumen of the access catheter and to be introduced with the catheter through the radially expanded lumen of the radially expandable sheath and into the patient's vasculature. In other embodiments, the percutaneous access systems of the present invention may be used with standard access catheters and/or guidewires which are not part of the system.

In some embodiments, the percutaneous access systems of the present invention further comprise a splittable proximal hub having a hemostatic valve secured to a proximal end of the radially expandable sheath. In such embodiments, the radially expandable sheath may be axially scored along one side to form a splitable seam. Often, the splittable seam on the sheath is aligned with a fracture line or region formed on the splittable proximal hub so that splitting of the hub will propagate splitting of the sheath along its seam.

In preferred embodiments, the radially expandable sheath comprises a tubular body having at least one axially aligned, everted fold in its radially constricted configuration, often having two, three, or more of such folds. The folds may be formed as follows. An axially aligned circumferential section of the tubular body is first compressed or “pinched” together to form a radially outwardly extending axial crease, and the crease may then be folded over onto an adjacent outer surface of the sheath to retain the generally tubular or cylindrical geometry with a reduced width or diameter. In some embodiments, the folds may be layer over one another.

In exemplary embodiments, the radially expandable sheath may comprise tubing formed from one or more polymers selected from a group consisting of fluorinated ethylene propylene (FEP), polytetrafluoroethylene (PTFE), ethylene tetrafluoroethylene (ETFE), polyoxymethylene (POM), polybutylene terephthalate (PBT), polyether block ester, polyurethane, polypropylene (PP), polyethylene (PE), low density polyethylene (LDPE), high density polyethylene (HDPE), linear low density polyethylene (LLDPE), polyester, polyamide, elastomeric polyamides, silicones, poly-ethylene terephthalate (PET), polyetheretherketone (PEEK), polyimide (PI), polyetherimide (PEI), polyphenylene sulfide (PPS), polyphenylene oxide (PPO), polysulfone, nylon. In preferred instances, the radially expandable sheath comprises tubing comprising, consisting essentially of, or consisting of fluorinated ethylene propylene (FEP).

In some examples, the tubing may have a wall thickness in a range from 0.01 mm to mm. In some examples, the polymer may have a density in a range from 2 g/cm³ to 2.2 g/cm³, an elongation-at-break above 200%, typically in a range from 200% to 400%, a flexural modulus in a range from 100 MPa to 1000 MPa, and/or a tensile strength in a range from 2000 MPa to 4000 MPa. In other instances the flexural modulus may be in a range from 100 MPa to 1000 MPa and the tensile strength may be in a range from 0.25 GPa to 1 GPa.

In a second aspect, the present invention provides a method for placement of an access catheter through a tissue access tract into a patient's vasculature, usually the venous vasculature but sometimes the arterial vasculature. The method comprises percutaneously inserting a distal portion of a radially expandable sheath into a blood vessel in a patient's vasculature. A dilator is introduced through a lumen of the radially expandable sheath to effect radial expansion of a lumen of the sheath. The dilator is removed with the radially expandable sheath maintaining its radially expanded configuration. A catheter is introduced through the expanded lumen of the radially expandable sheath so that a distal end of the catheter reaches a lumen of a target blood vessel in the venous or other vasculature. The radially expanded sheath is then split along an axial seam line; and removed from over the catheter, leaving the catheter in place for long term use.

In some embodiments, the methods of the present invention further comprise confirming that the distal portion of the radially expandable sheath has entered the blood vessel. Confirming may comprises ultrasonic imaging, observing blood flashback, or any other known techniques.

In some embodiments, the dilator expands the radially expandable sheath into a cylindrical configuration which is maintained after removal of the dilator. Usually, the radially expanded cylindrical sheath has an inner diameter that is no more than 1 mm greater than an outer diameter of the catheter.

In preferred instances, the catheter carries a guidewire in a guidewire lumen of the catheter when the catheter is introduced though the expanded sheath lumen and into the blood vessel. In such instances, the methods usually further comprise advancing the guidewire from the guidewire lumen of the catheter to a target location in the patient's vasculature after the catheter has been introduced though the expanded sheath lumen and into the blood vessel. The catheter is typically advanced over the guidewire to the target location in the patient's vasculature after advancing the guidewire has been advanced from the guidewire lumen of the catheter.

The methods of the present invention may be used with any venous, arterial, or other target vessel but will find particular use with is any one of an internal jugular vein, a subclavian vein, an axillary vein, or a femoral vein.

In a third, alternative aspect, the present invention provides a percutaneous access system for placement of a vascular catheter through a tissue access tract into a patient's vasculature that does not require removal of a dilator prior to advancement of the catheter into the patient's vasculature. Eliminating the dilator removal step is advantageous as it reduces the time and steps necessary to perform the access procedure.

An example of such an alternative system comprises an access needle, a radially expandable sheath, and a carrier dilator. The access needle may have a conventional design with a sharpened distal tip with a proximal hub having a luer or other connector. The proximal hub of the access needle is optionally configured to mate with a needle guide as describe elsewhere herein.

The radially expandable sheath comprises a tubular sheath body having a distal end, a proximal end, and a sheath lumen therebetween. The tubular sheath body is configured to be radially expanded from a radially constricted configuration to a radially expanded configuration, and the sheath lumen is typically configured to receive the access needle when said sheath body is in its radially constricted configuration. In most instance, the tubular sheath body will be either split or splittable, typically along a single axial line, to allow it to be proximally retracted and removed from over a vascular catheter after the catheter has been positioned in a tissue tract.

The carrier dilator comprises a tubular carrier body having a distal end, a proximal end, and a carrier lumen therebetween. The carrier lumen is configured to carry the vascular catheter, and the distal end the tubular carrier body is tapered to dilate the tubular sheath body as the carrier dilator is advanced through the sheath lumen to expand the sheath body from its radially constricted configuration to its radially expanded configuration. In most instance, the tubular carrier body will be either split or splittable, typically along a single axial line, to allow it to be proximally retracted and removed from over a vascular catheter after the catheter has been positioned in a tissue tract.

Use of a “carrier dilator” allows tissue tract dilation and catheter introduction steps to be performed in a combined step which reduces both the complexity and duration of the access protocol. The complexity and duration of the access protocol may be further reduced by pre-loading a guidewire in the access catheter as will be described in greater detail below.

In some examples, the radially expandable sheath further comprises a sheath proximal hub.

In some examples, the carrier dilator further comprises a carrier proximal hub configured to detachably couple to the sheath proximal hub.

In some examples, the sheath proximal hub and the carrier dilator hub are split or splittable individually and when coupled to allow removal over the vascular catheter as will be described in more detail below. In such instances, the split or splittable structure in each hub will be aligned with a split or splittable feature, e.g. a break or a score line” formed on the tubular sheath or carrier body, so that the assembly of the radially expandable sheath and the carrier dilator can be removed in a single step after splitting the hubs.

In some examples, the tubular sheath body and the tubular carrier body are split or splittable to allow removal over the vascular catheter as will be described in more detail below.

In some examples, the percutaneous access system further comprising a needle guide configured to be removably placed in the sheath proximal hub and having a central passage which guides a distal end of the access needle into the sheath lumen. In addition to facilitating access needle introduction, the needle guide may include an insert portion that supports a tapered transition region of the radially expandable sheath between the sheath hub and the radially constricted portion of the tubular sheath body.

In some examples, percutaneous access system further comprises the vascular catheter.

In some examples, the percutaneous access system further comprises a guidewire configured to be received in a guidewire lumen of the vascular catheter. The guidewire will usually be introduced with the catheter into the tubular body of the carrier dilator, but in other instances could be introduced before or after the vascular catheter.

In some examples, the radially expandable sheath body comprises tubing formed from one or more polymers selected from a group consisting of fluorinated ethylene propylene (FEP), polytetrafluoroethylene (PTFE), ethylene tetrafluoroethylene (ETFE), polyoxymethylene (POM), polybutylene terephthalate (PBT), polyether block ester, polyurethane, polypropylene (PP), polyethylene (PE), low density polyethylene (LDPE), high density polyethylene (HDPE), linear low density polyethylene (LLDPE), polyester, polyamide, elastomeric polyamides, silicones, poly-ethylene terephthalate (PET), polyetheretherketone (PEEK), polyimide (PI), polyetherimide (PEI), polyphenylene sulfide (PPS), polyphenylene oxide (PPO), polysulfone, nylon.

In some examples, the radially expandable sheath body comprises tubing consists essentially of fluorinated ethylene propylene (FEP).

In some examples, the tubing has a wall thickness in a range from 0.01 mm to 0.2.

In some examples, the polymer has a density in a range from 2 g/cm³ to 2.2 g/cm³, an elongation-at-break above 200% in a range from 200% to 400%, a flexural modulus in a range from 100 MPa to 1000 MPa, a tensile strength in a range from 0.25 GPa to 1 GPa.

The percutaneous access systems as just described may be used in methods for placing a vascular catheter through a tissue access tract into a patient's vasculature using the “carrier dilator” to eliminate the need for separate dilation and catheter introduction steps. A distal end of a tubular sheath body of a radially expandable sheath is percutaneously inserted into a blood vessel in the patient's vasculature while the tubular sheath body is in a radially constricted configuration. A tubular carrier body of a carrier dilator is advanced through a lumen of the tubular sheath body causing the tubular sheath body to radially expand from its radially constricted configuration to a radially expanded configuration. A distal portion of the vascular catheter is disposed in a lumen of the tubular body of the carrier dilator while the tubular carrier body is being introduced, eliminating the need to separately introduce the vascular catheter after removing the dilator, as described for example in connection with the first system embodiment described herein. Typically, the tubular carrier body of the carrier dilator has a tapered distal tip (to facilitate advancement through and expansion of the sheath lumen) which is opened by advancement of the vascular catheter through the tubular carrier body of the carrier dilator, as described in more detail hereinbelow. In some instances, a distal tip of the vascular catheter will extend distally of a distal tip of the tubular carrier body of the carrier dilator as the carrier dilator is being advanced. In other instances, the distal tip of the vascular catheter will be retracted within a distal portion of the tubular carrier body as the carrier dilator is being advanced.

In some examples, such methods further comprise advancing the distal portion of the vascular catheter from the lumen of the tubular body of the carrier dilator into the blood vessel while the distal end of the tubular sheath body of the radially expandable sheath remains in the blood vessel.

In some examples, such methods further comprise axially splitting the radially expanded sheath and the carrier dilator after the distal portion of the vascular catheter has been advanced into the blood vessel and removing the axially split radially expanded sheath and the axially split carrier dilator from over the vascular catheter while the distal portion of the vascular catheter remains in the blood vessel.

In some examples, the radially expanded sheath and the carrier dilator are axially split and removed simultaneously.

In some examples, a sheath proximal hub on the radially expandable sheath and a carrier proximal hub on the carrier dilator are coupled together after the tubular carrier body of the carrier dilator has been advanced through the lumen of the tubular sheath body and before the radially expanded sheath and the carrier dilator are axially split.

In some examples, such methods further comprise confirming that the distal portion of the radially expandable sheath has entered the blood vessel prior to advancing the tubular carrier body of the carrier dilator through the lumen of the tubular sheath body.

In some examples, confirming comprises ultrasonic imaging.

In some examples, confirming comprises observing blood flashback.

In some examples, the vascular catheter carries a guidewire in a guidewire lumen thereof when the distal portion of the vascular catheter is carried in the tubular carrier body.

In some examples, such methods further comprise advancing the guidewire from the guidewire lumen of the vascular catheter before or after (usually after) the tubular catheter body has been advanced into the vasculature.

In some examples, such methods further comprise advancing the catheter over the guidewire to a target location in the patient's vasculature after the guidewire has been advanced.

In some examples, the blood vessel may be any one of an internal jugular vein, a subclavian vein, an axillary vein, or a femoral vein.

In some examples, percutaneously inserting the distal end of the tubular sheath body of the radially expandable sheath into the blood vessel comprises placing an access needle into the lumen of the tubular sheath body so that a sharpened tip of the needle extends distally of the distal end of the tubular sheath body and simultaneously inserting the access needle and the radially expandable sheath.

In some examples, advancing the access needle into the lumen of the tubular sheath body comprises attaching a needle guide to a proximal end of the sheath lumen and inserting the access needle through the needle guide.

In some examples, such methods further comprise loading a guidewire into a guidewire lumen of the vascular catheter prior to disposing the distal portion of the vascular catheter into the lumen of the tubular body of the carrier dilator.

Although described with reference to particular catheters in the drawings herein, the systems and methods of the present invention are useful for placing a variety of indwelling medical devices, such as sheaths, midlines, dialysis catheters, and arterial lines, as well as central venous catheters (CVCs), peripherally inserted central catheters (PICCs), peripherally inserted venous catheters (PIVCs), and the like.

### BRIEF DESCRIPTION OF THE DRAWINGS

FIG. 1. summarizes the steps performed in the commonly used Seldinger technique of the prior art.

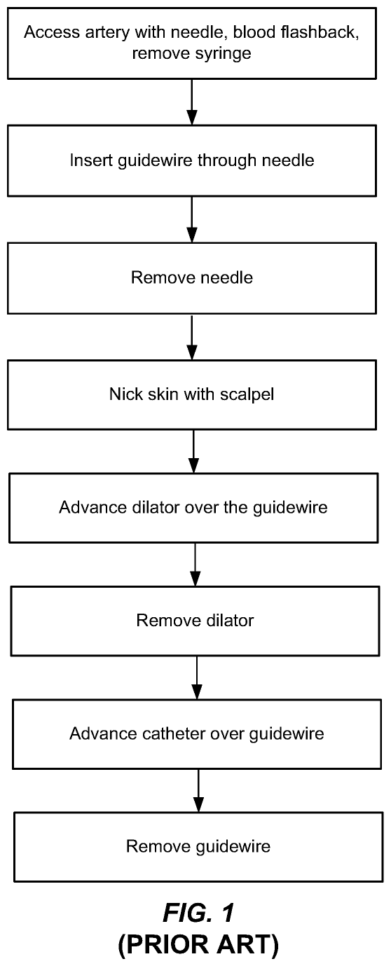

FIG. 2 provides images the steps of the Seldinger technique showing where the prior art devices can make skin contact increasing the risk contamination.

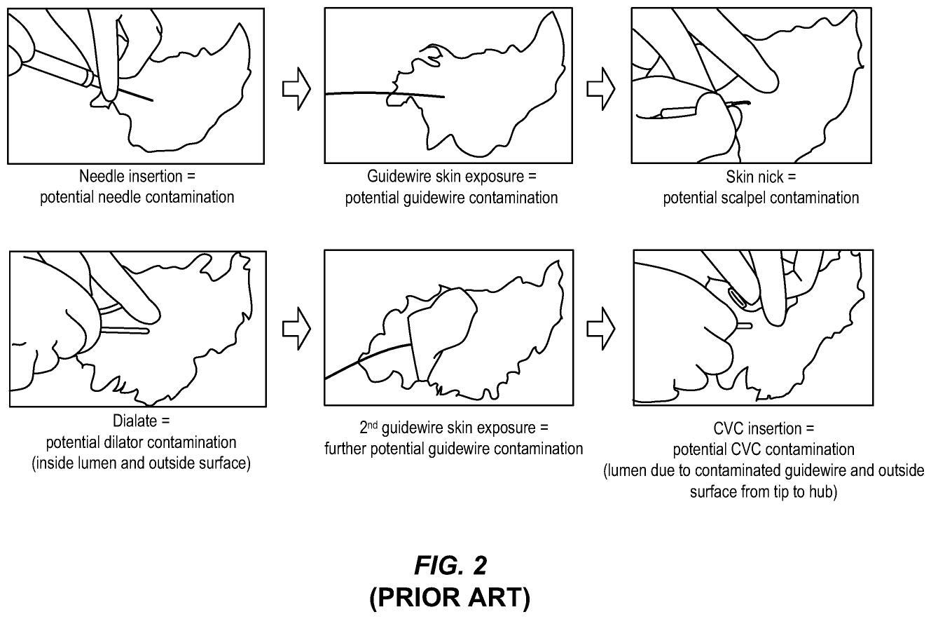

FIG. 3 summarizes the steps in the methods of the present invention.

FIG. 4 is an isometric view of a radially expandable sheath and access needle assembly constructed in accordance with the principles of the present invention.

FIG. 5 separately illustrates the radially expandable sheath and the access needle of FIG. 4 together with a dilator.

FIG. 6 is an enlarged, cross-sectional view of a splittable hub at the proximal end of the radially expandable sheath of FIG. 4.

FIGS. 7A-7C are alternative cross-section view taken along line 7-7 in FIG. 4.

FIG. 8 illustrates the Radially expandable heath of FIGS. 4 and 5 shown in its radially expanded configuration after having been expanded by the dilator of FIG. 5.

FIGS. 9A-9J illustrate an exemplary percutaneous venous access procedure performed in accordance with the principles of the present invention.

FIG. 10 is an exploded view showing the components of a second exemplary embodiment of a percutaneous access system constructed in accordance with the principles of the present invention.

FIG. 11 illustrates an assembly of an access needle, a needle guide, and a radially expandable sheath which are three components of the percutaneous access system of FIG. 10.

FIG. 12 is a detailed view of the proximal end of the assembly of FIG. 11 shown in partial cross-section with the needle removed.

FIG. 13 illustrates an assembly of a radially expandable sheath, a carrier dilator, and a vascular catheter which are three components of the percutaneous access system of FIG. 10.

FIG. 14A is detailed view of the distal end of the assembly of FIG. 13.

FIG. 14B is detailed view similar to that of FIG. 14A shown with a distal end of the vascular catheter fully advanced through a distal end of the carrier dilator.

FIG. 14C is a distal end view of the distal end of the assembly of FIG. 13 showing a gap that allows the vascular catheter to advance freely within the radially expandable sheath after the sheath has been expanded.

FIGS. 15A and 15B are enlarged, end views of a splittable hub assembly at the proximal ends of the radially expandable sheath and the carrier dilator, with FIG. 15A showing the hub assembly in its closed (pre-split) configuration and FIG. 15B showing the hub assembly in its open (post-split) configuration.

FIG. 16 is a further view of the splittable hub assembly as shown in FIGS. 15A and 15B.

FIGS. 17A-17F illustrate a second exemplary percutaneous vascular access procedure performed in accordance with the principles of the present invention.

### DETAILED DESCRIPTION OF THE INVENTION

The systems and methods of the invention provide improvements on the Seldinger technique, as described above, through use of an expandable, peel-away radially expandable sheath, referred to hereinafter and in the claims as a “radially expandable sheath,” typically having a splittable proximal hub that can include a hemostatic valve. The radially expandable sheath typically carries a removable access needle during the initial puncture, and the radially expandable sheath is expanded using a dilator which is the removed to provide a sterile conduit for introduction of the access catheter to the patient's vasculature without touching the skin and minimizing the chance of contamination. This method reduces the number of skin-instrument interactions to only one, the initial puncture, significantly reducing the risk of bacteria contaminating the tissue tract and/or the catheter. Moreover, dilation within the lumen of the radially expandable sheath reduces the risk of injuring tissue surrounding the tissue tract by lessening the axial forces on the tissue. Additionally, use of a hemostatic valve reduces or eliminates the risk of open-to-air events.

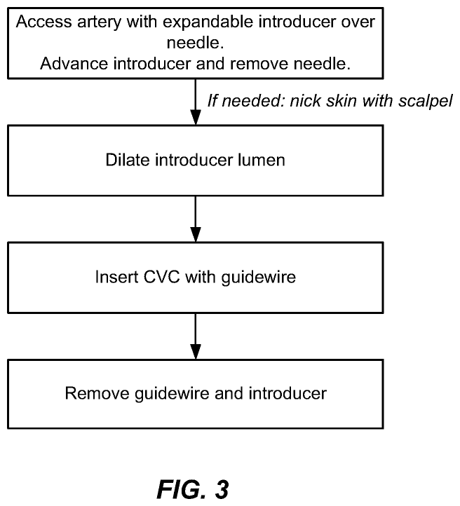

As summarized in FIG. 3, the radially expandable sheath is placed over the access needle and the assembly of the radially expandable sheath and the access needle is used to create a tissue tract to access the a target blood vessel in the patient's venous or other vasculature. Upon entering the target blood, the radially expandable sheath is advanced forward into a lumen of the blood vessel, and the access needle is withdrawn from the radially expandable sheath after access has been confirmed, typically by observing flashback or by ultrasonic imaging, e.g., the radially expandable sheath can be echogenic ally enhanced and introduction can be performed under ultrasonic imaging to verify the radially expandable sheath is in the target vessel. If ultrasound is unavailable, a pressure check can be performed by an additional pressure check device included in the kit, or one that is integrated in the hub. Venous or arterial pressure may be assessed.

After the target vessel access has been confirmed, the radially expandable sheath is expanded utilizing a tapered dilator to an inner lumen diameter roughly the size of the catheter to be placed. The radially expandable sheath will be constructed so that dilation by the dilator will not split the lumen. The dilator is removed from the radially expandable sheath after radial expansion, and the catheter (typically with a pre-loaded J-tipped or other guidewire for support) is inserted through the expanded radially expandable sheath lumen and typically a hub and hemostatic valve at the proximal end of the sheath. The catheter is advanced to the distal end of the radially expandable sheath lumen, and the guidewire is advanced further (if needed) to guide the catheter as it continues to be advanced through the vasculature. Alternatively, although generally less preferred, the guidewire may be introduced through the expanded radially expandable sheath and the catheter then advanced over the guidewire through the expanded radially expandable sheath.

Once the access catheter is in position, the radially expanded sheath is split and peeled away (separated) while maintaining the position of the catheter. Typically, the radially expanded sheath is split by first splitting an attached proximal hub, as with a conventional peel-away sheath. The guidewire may then be withdrawn from the catheter, and vascular access is established.

When using the percutaneous access system of the present invention, the guidewire, dilator and dwelling catheter never touch the skin or tissue tract, avoiding potential bacterial contamination from the skin, the primary source of infection. This method of the present is invention is also simpler, has fewer steps, and can minimize or avoid the use of difficult-to-control components, such as a floppy guidewire, as well as minimizing the risk of losing the guidewire in the patient.

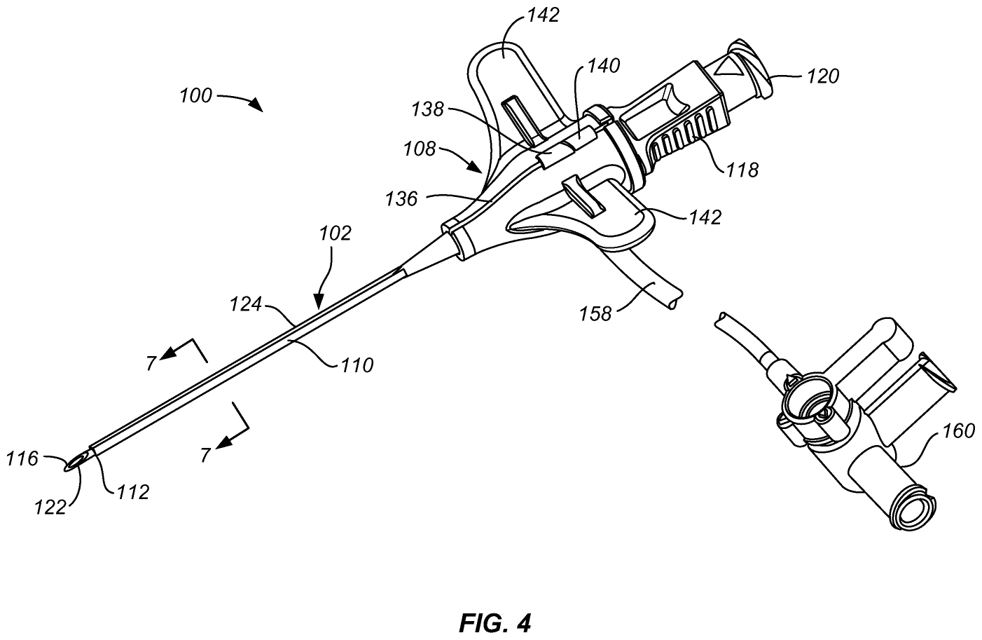

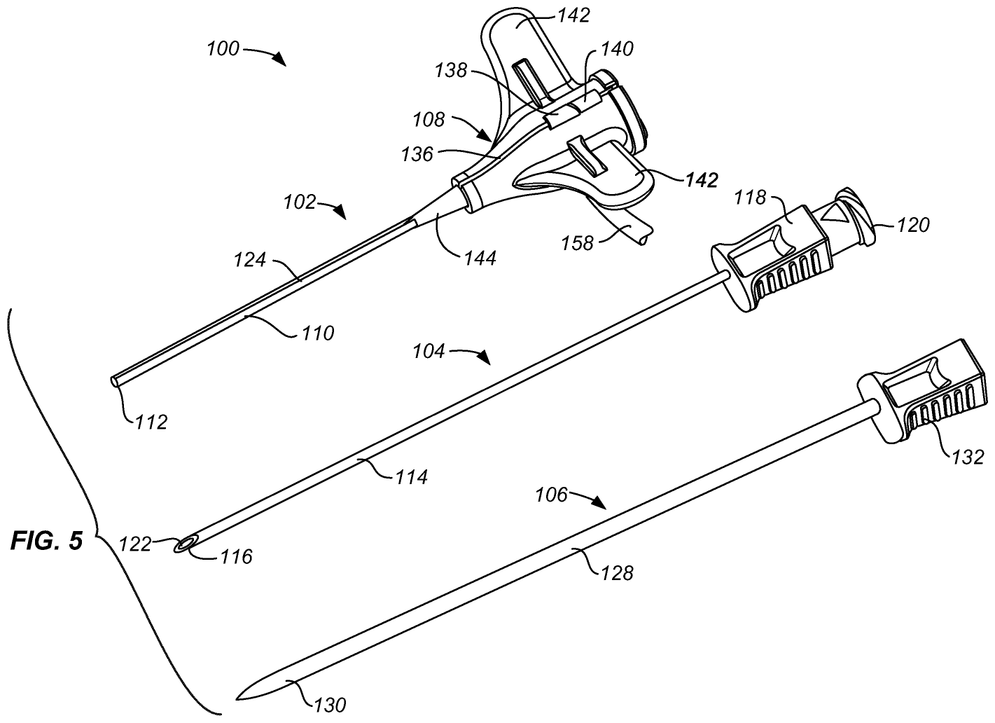

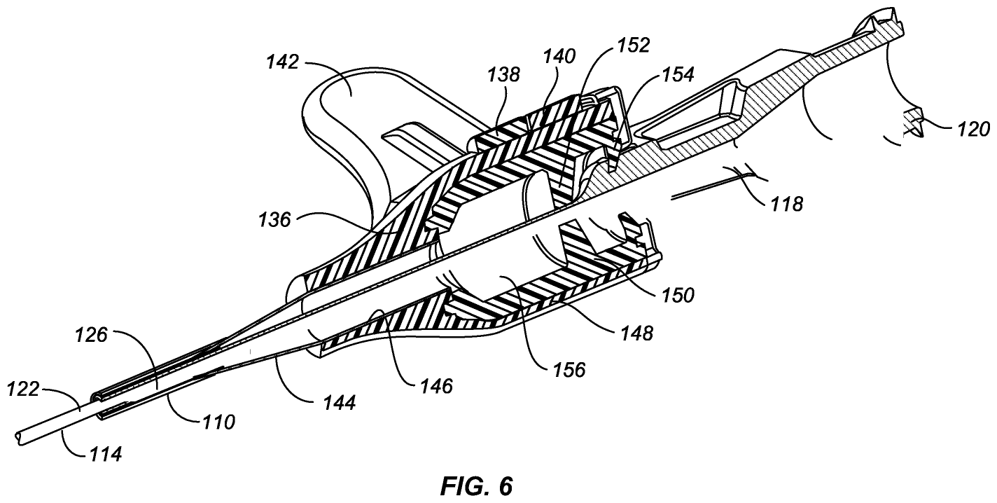

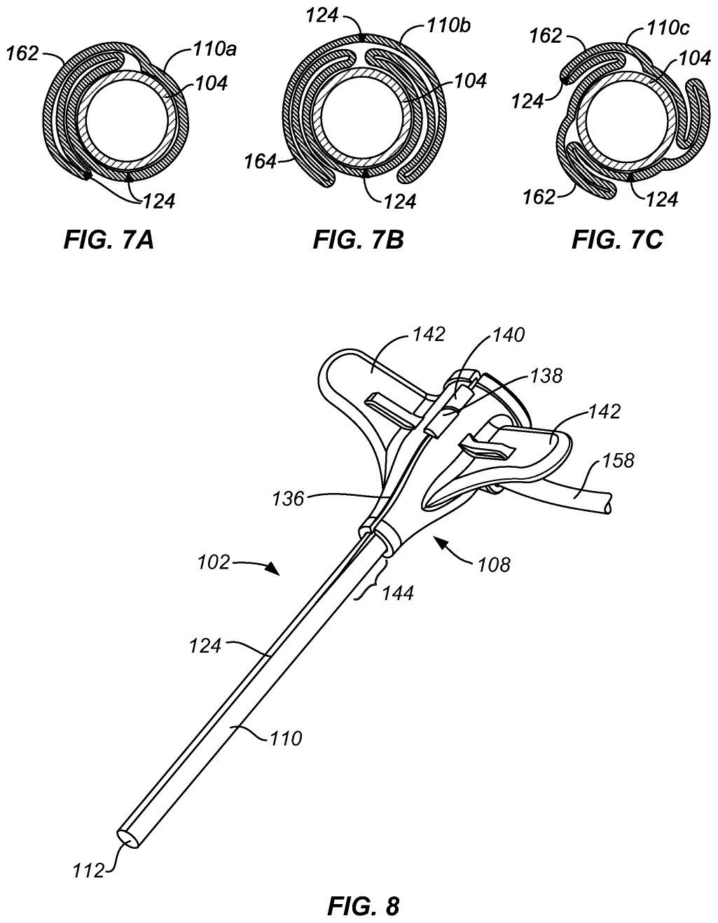

Referring now to FIGS. 4 to 6, a percutaneous access system 100 comprises a radially expandable sheath 102, an access needle 104, and a dilator 106. The radially expandable sheath 102 has a proximal hub 108, a tubular member 110, and a distal opening 112. The tubular member 110 is typically formed a polymer tube, as described in greater detail below, having an inner lumen 126 (FIG. 6) and a pair of score lines 124 axially inscribed along opposite sides thereof (as best seen in FIGS. 7A-7C). The score lines 124 are intended to allow splitting of the tubular member 110 when the radially expandable sheath 102 is being removed. The proximal hub 108 is also splittable, typically along an axial split 136 formed in an upper surface of the hub (as viewed in FIG. 6) and a weakened line 148 formed axially along a lower surface of the hub. Splitting is accomplished by pushing in opposite lateral directions on the tabs 138 and 140. The score lines 124 will be online with the axial split 136 and weakened region 148, respectively, so that splitting the hub will pull apart the tubular member 110 of the radially expandable sheath when the sheath is being withdrawn. The proximal hub 108 includes wings 142 which facilitate manipulation by the user. Although shown to split into halves, in other instances the hub maybe split in one plane, multiple planes, or in other patterns to allow removal over the access catheter and catheter hub.

The access needle 104 includes a needle shaft 114 having a tissue-penetrating tip 116 and an inner needle lumen 122. The inner needle lumen 122 extends from the tip 116 to a luer connector 120 in a proximal hub 118. The construction of the needle is conventional and allows blood to flow from the tip 116 to the luer connector 120 when the needle tip enters a blood vessel allowing blood flashback.

The dilator 106 is also conventional and includes a dilator shaft 128, a tapered distal tip 130, and a proximal dilator hub 132. As will be described in greater detail below, the tapered distal tip 130 allow the dilator 108 to be advanced through the tubular member 110 of the radially expandable dilator 102, causing the tubular member to radially expand without splitting along the score lines 124.

Referring further to FIG. 6, the splittable proximal hub 108 of the radially expandable sheath 102 is attached to the tubular member 110 by a conical transition region 144. The transition region 144 has a proximal end which is contiguous with a radially enlarged attachment region 146 at a proximal end of the tubular member 110, where the attachment region has a width or a diameter which is equal to or slightly larger than the size of the sheath lumen 126 when fully expanded. In contrast, the distal portion of the tubular member 110 which is radially constricted has a width or diameter which is equal to or only slightly larger than the outer diameter of the needle shaft 114 prior to radial expansion. The radially enlarged attachment region 110 of the tubular member is secured in a cavity formed in the distal end of the proximal hub 108.

The proximal hub 108 includes a hemostatic valve insert 150 which includes a pair of axially spaced-apart slit valves 152 and 154 which together allow insertion and removal of the access needle 104, the dilator 106, and the venous access catheter while minimizing blood loss. The hemostatic valve insert 150 further includes a chamber 156 which collects blood after open end 112 of the radially expandable sheath 102 enters a blood vessel. The chamber 156, in turn is connected to a pressure check tube 158 which terminates in an on-off valve 160. Alternatively, a pressure checking feature could be incorporated into the proximal hub 108.

Referring now to FIGS. 7A-7C, the tubular member 110 of the radially expandable sheath 102 may be constricted to receive the needle shaft 104 in a variety of ways. For example, wall of the radially expandable sheath 102 in its radially constricted configuration may have one or more folds so that upon dilator insertion and advancement, the lumen unfolds and increases in diameter to accommodate the dwelling device (e.g. a venous or other access catheter). The advantage to the folds is that upon dilation, the material unfolds with less force compared to stretching a polymer material. The inside lumen wall may be flat or incorporate axial ribs that are slightly raised to reduce the force required to expand the lumen by minimizing the contact surface area and friction between the dilator and inside lumen wall. The lumen wall after expansion will have a diameter just larger than the dilator.

The dilator 106 diameter can be smaller or larger than the catheter or other device the radially expandable sheath 102 and will accommodate a partial or full expansion without splitting the expanded tubular body. The lumen wall may have one or more pre-defined axial lines that are weaker to facilitate the peel-away or separation of the radially expandable sheath 102 after the catheter or other device has been advanced. Such axial separation lines can be scored (by razor blade, laser, etc.) to a uniform pattern or dashed pattern with various depths to facilitate a consistent and easy peel when removing the radially expandable sheath. As an alternative to pre-scoring, the catheter hub may have an integrated cutting/splitting feature in the hub that splits the lumen as the expandable radially expandable sheath is withdrawn over the catheter hub.

As shown in FIG. 7A, the tubular member 110 has a single everted fold 162 which is wrapped back over remaining cylindrical portion of the tubular member. Axial score lines 124 are show adjacent to each other when the tubular member is constricted but will open to diametrically opposed positions when the tubular member is radially expanded. As shown in FIG. 7B, an everted double fold 164 has a pair of flaps which are wrapped back over opposite sides of the tubular member when constricted. Axial score lines 124 are initially opposed to each other and remain so after the sheath is radially expanded. A third configuration as shown in FIG. 7C where three everted fold lines 162 are located at 12 o'clock, 4 o'clock, and 8 o'clock about the periphery of the tubular member 110. The resulting three flaps are folded over and shown not to overlap. In other instances, the flaps resulting from one or more everted fold lines could overlap one another.

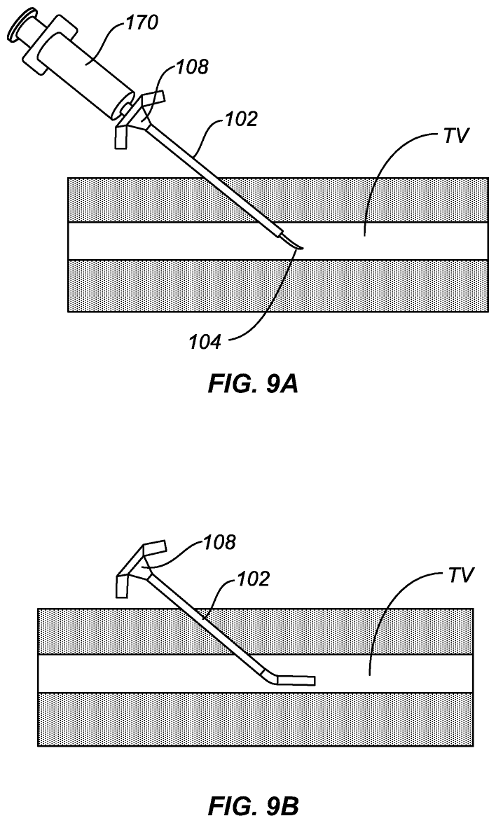

Referring now to FIG. 8, the radially expandable sheath 102 is shown with the tubular member 110 in its radially expanded configuration after advancement and removal of the dilator 106 but prior to splitting along score lines 102. The radially expandable sheath 102 is in a configuration ready to receive an access catheter for introduction into the patient's vasculature.

In some instances, the distal end of the radially expandable sheath may be tapered (e.g. by heat/radiofrequency forming) to provide a smooth transition into the access needle tip. The tubular member 110 of the radially expandable sheath 102 is typically made of a polymeric material that may comprise, consist essentially of, or consist of fluorinated ethylene propylene (FEP)polytetrafluoroethylene (PTFE), ethylene tetrafluoroethylene (ETFE), polyoxymethylene (POM), polybutylene terephthalate (PBT), polyether block ester, polyurethane, polypropylene (PP), polyethylene (PE), low density polyethylene (LDPE), high density polyethylene (HDPE), linear low density polyethylene (LLDPE), polyester, polyamide, elastomeric polyamides, silicones, poly-ethylene terephthalate (PET), polyetheretherketone (PEEK), polyimide (PI), polyetherimide (PEI), polyphenylene sulfide (PPS), polyphenylene oxide (PPO), polysulfone, nylon, or other suitable materials, or mixtures, combinations, copolymers thereof, polymer metal composites, and the like. The lumen material may have a hydrophilic coating or loaded with an echogenic material (e.g. hollow glass beads, etc.) and/or a radiopaque material (barium sulfate, etc.).

The pressure check tubing 158 is typically integrated into the hub. The tubing comes out of the hub (distal to the hemostatic valve) and includes an on/off or pressure check valve at the end. When the valve is opened, this tubing functions as a manometer to visualize pressure of the vein/artery where the radially expandable sheath resides. A pressure transducer may also be connected to the luer-lock or luer-slip style connector on the on/off valve.

An alternative to the integrated pressure check tubing described above, the system or kit may include a flexible thin-walled non-collapsible tube having a diameter similar or identical to the access needle diameter with a length that can range from short (just breaching the hemostatic valve) to long (length reaching the distal tip of the radially expandable sheath lumen). This pressure check tubing may be connected to a flexible clear tube of larger diameter that is proximal to the hemostatic valve and functions as a clear manometer to visualize the blood pressure. This larger diameter tube has a luer-lock or luer-slip style connector on the end to connect to a pressure transducer.

The access catheter may be included as part of the system or kit may and may wholly or partially packaged a separate sterile bag. The sterile thin film bag is bonded/fused to the catheter hub so when it is gently tugged, it separates from the hub. The bag also includes one or more axial perforated folds. When the catheter is advanced into the radially expandable sheath hub, the bag bunches together axially and can be separated from the catheter using the perforations and tear away bond at the catheter hub. The bag serves as an added layer of protection from potential bacterial contamination on a clinician's gloves during insertion.

The present invention provides kits for performing the methods described. The kits may contain an expandable and peel-away radially expandable sheath with a splittable hub that can include a hemostatic valve, a pressure check device, a dilator, a catheter or sheath, syringe, access needle, scalpel, and a catheter reinforcing wire. The kit may additionally contain all the accessories (skin prep, drapes, etc.) to complete the vascular access procedure from start to finish.

In one example, the expandable, splittable sheath may be formed from a material that can initially be folded or furled into a constricted, small diameter configuration which can be expanded by the advancement of a tapered or cylindrical dilator through a lumen thereof. The material will be foldable and shapeable but will have sufficient wall stiffness and hoop strength to resist cylindrical stress applied by the wall of the tissue tract in which the sheath was expanded.

High density polyethylene (HDPE) is one suitable material having a durometer/stiffness providing hoop strength when unfolded. HDPE with a wall thickness in a range from 0.01 mm to 0.2 mm, with a value of 0.075 mm (0.003 in) having been suitable. The natural lubricity of HDPE helps reduce friction between the outer lumen surface/tissue when advancing the radially expandable sheath over the access needle into the vessel and also when the dilator and catheter are advanced through the lumen.

The radially expandable sheath may be fabricated by pulling extruded HDPE tubing through a heated cylindrical metal die. The dye is configured to fold and wrap the tube over a mandrel as the tube moves through the heated dye. The compressed tube is then “set” in an oven to maintain its shape. The heated metal die compresses the tube radially over a mandrel to create folds/pleats. The second step uses radial compression of a heated die to wrap the folds in a manner similar to folding and wrapping an angioplasty balloon on a catheter.

Prior to folding, a split line may be formed in the extruded HDPE tubing using conventional mechanical (e.g., razor) scoring, laser scored, or the like. In some instances, no score line may be needed due to materials that have inherent tear properties.

After the dilator is removed from the expanded radially expandable sheath, it is not critical that the expanded lumen maintain the full (100%) dilated diameter. The unfolded “expanded” lumen may undergo limited constriction, typically less than 25%, Usually less than 15%, and preferably less than 10% of the fully expanded diameter due to tissue compression. The lumen will typically expand as necessary to accommodate the catheter as it is inserted and advanced.

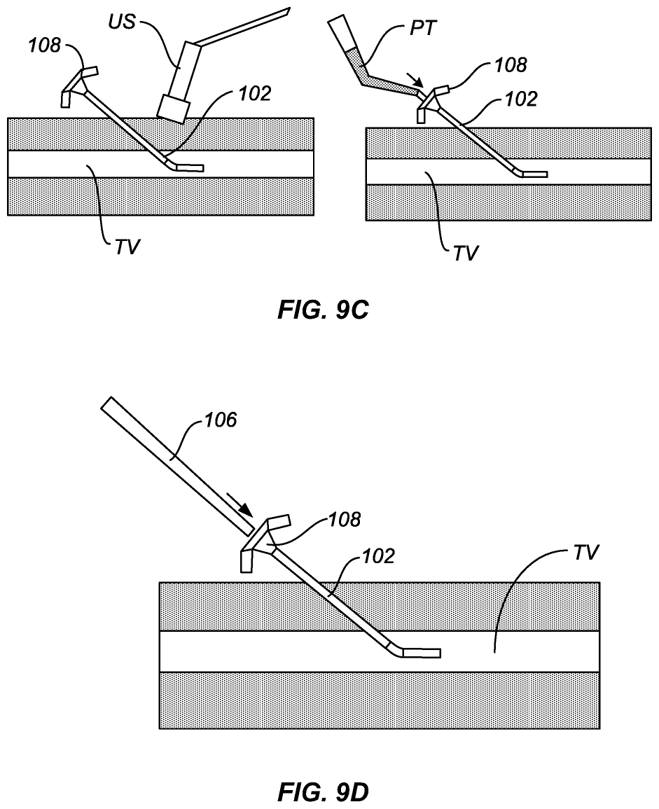

FIG. 9A illustrates accessing a target vasculature TV using an assembly including the access needle 104 and radially expandable sheath 102 of the present invention with a syringe 170 attached to splittable hub 108 that can include a hemostatic valve.

FIG. 9B illustrates the radially expandable sheath 102 advanced into the target vasculature TV with the needle withdrawn.

FIG. 9C (left panel) illustrates verification that the open distal end 112 of the radially expandable sheath 102 has entered in the target vasculature TV using a handheld ultrasound imaging device US. If ultrasound is available, the radially expandable sheath can be verified due to the echogenic properties of the lumen. If ultrasound is not available, pressure may be assessed via a check valve in the hub or tubing used as a manometer or connected to a pressure transducer PT (right panel).

FIG. 9D illustrates the dilator being advanced into the lumen 126 (FIG. 6) of the radially expandable sheath 102.

FIG. 9E illustrates the dilator 106 expanding the lumen 126 of the radially expandable sheath 102 to approximately the diameter of the catheter or other dwelling medical device to be introduced.

FIG. 9F illustrates the dilator 106 removed and the radially expandable sheath 102 in the target vasculature in the expanded state.

FIG. 9G illustrates an access catheter AC or other medical device carrying a guidewire GW being inserted into the expanded lumen of the radially expandable sheath 102.

FIG. 9H illustrates the catheter AC at a desired position in target vasculature TV with the guidewire GW at its distal end.

FIG. 9I illustrates the hub 108 of the radially expandable sheath 102 being split by pulling the wings 142 in the direction of the arrows. The radially expandable sheath is peeled away and pulled back leaving the catheter AC in place.

FIG. 9J illustrates the catheter in place and the guidewire GW being removed.

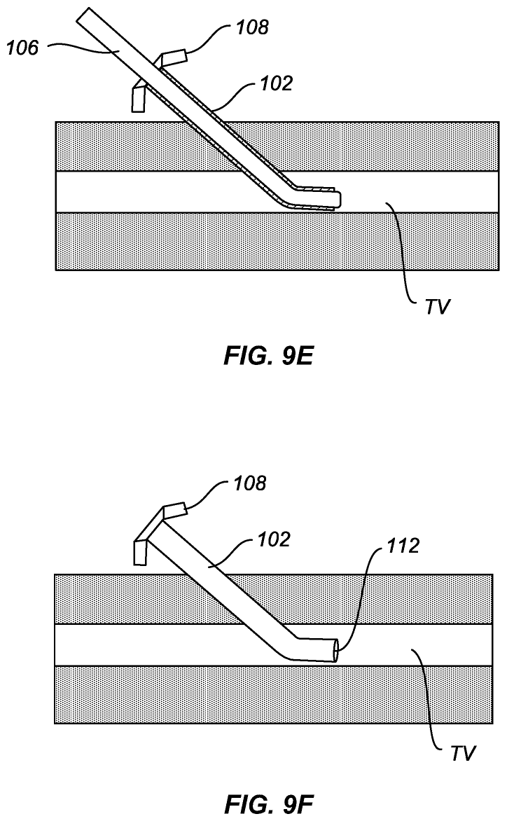

Referring now to FIG. 10, a second embodiment of a percutaneous access system 200 constructed in accordance with the principles of the present invention will be described. The percutaneous access system 200 includes a radially expandable sheath 202, a carrier dilator 204, a needle guide 206, an access needle 208 having a hub 242, and a vascular catheter 210 having a hub 262. While the radially expandable sheath 202, the carrier dilator 204, and the needle guide 206 will usually be designed specifically for use in the systems of the present invention, the access needle 208 and the vascular catheter 210 may at least sometimes be provided as “off the shelf” components. For example, at least the radially expandable sheath 202 and the carrier dilator 204 will usually be packaged together as a kit and be designed to work specifically with each other. The needle guide 206 will often but not necessarily be included as part of the kit, as the present invention can be practiced without using the needle guide, although its use is generally preferred. Conversely, the radially expandable sheath 202 and the carrier dilator 204 will usually be compatible with a wide variety of conventional access needles and vascular catheters which need not be part of the kits.

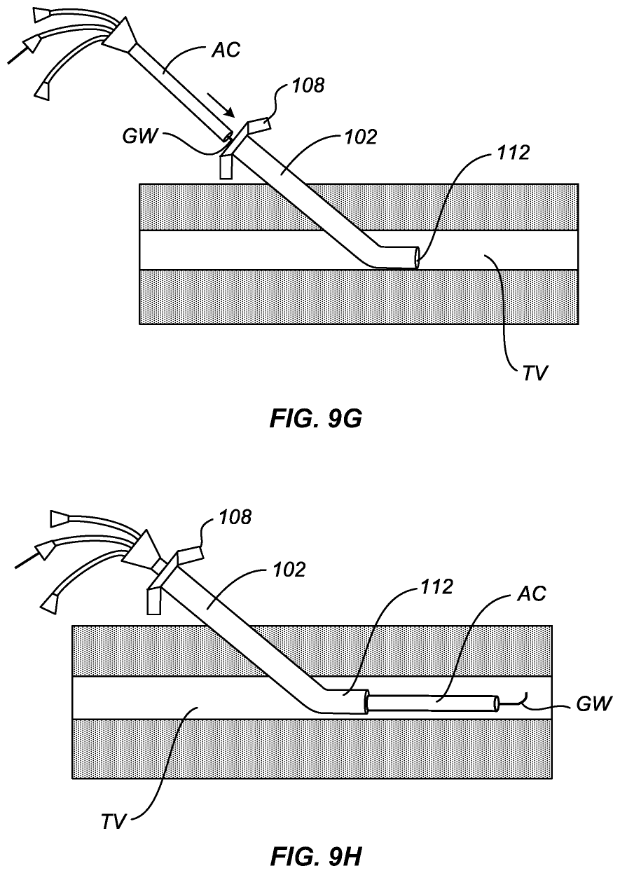

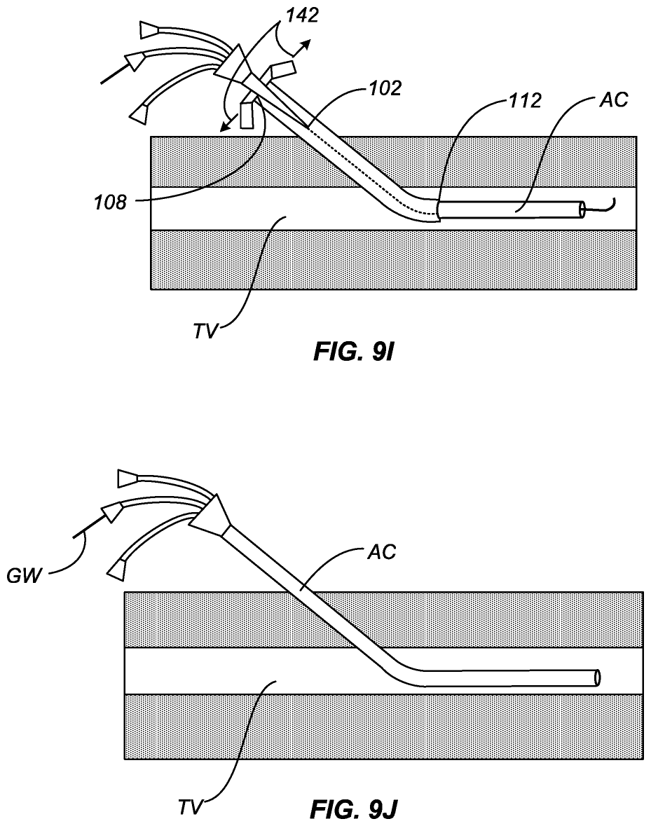

Referring now also to FIGS. 11 and 12, The radially expandable sheath 202 comprises a tubular sheath body 216 having a distal end 217 and a proximal hub 218. The proximal hub 218 includes a pair of “pull apart” wings 220 that allow the hub to be opened as will be described in greater detail below. In FIGS. 10 and 11, The tubular sheath body 216 is shown in a radially constricted configuration and is wrapped or folded so that it may be radially expanded by passage of the carrier dilator 204 through a lumen thereof, as will be described in detail below. The tubular sheath body 216 is further configured so that it is axially split or splittable to allow it be removed from over the catheter 210 after the catheter has been placed, as will also be described in greater detail below.

An insert portion 232 of the needle guide 206 is placed in the sheath proximal hub 218, as shown in FIG. 12, to facilitate introduction of the access needle 208. Usually, threads or snaps 254 on hub 234 of the needle guide 206 will be engaged with similar features on the hub 218 of the radially expandable sheath 202. A sharpened distal tip 240 of needle access tube 238 may be introduced through a needle access port 246 in the hub 242, and a tapered or funnel region 248 aligns the needle with passage 250 in the needle guide which in turn aligns the needle with a lumen of the tubular sheath body 216. A second taper region 252 may also be provided in order to enhance alignment. While the use of the needle guide 206 is not essential, it is advantageous as it reduces the risk of the needle damaging the tubular sheath body or other components of the system as it is advanced.

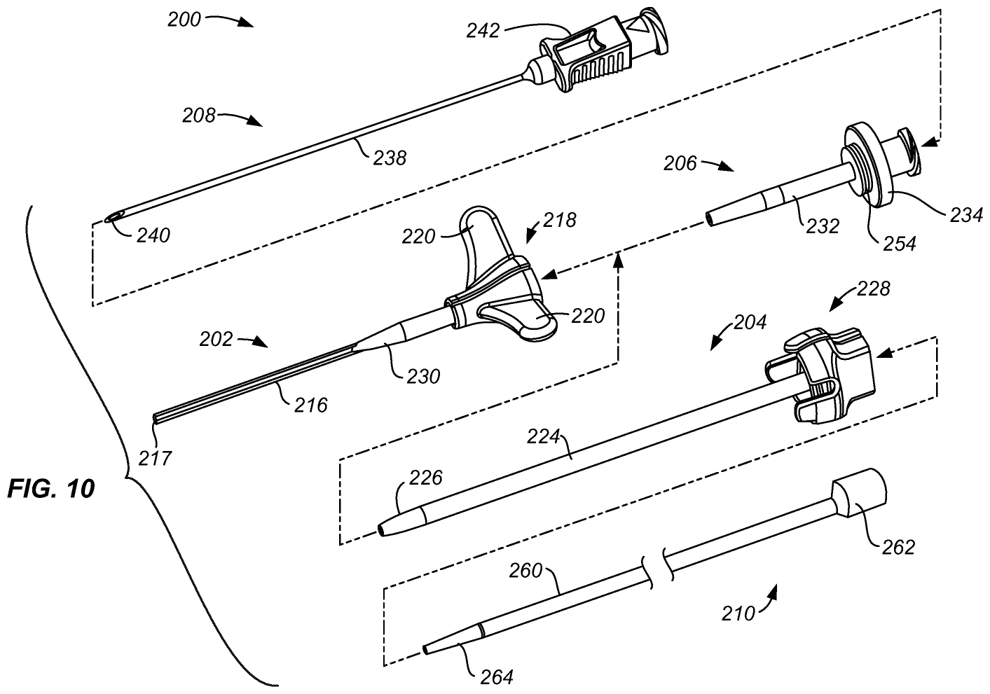

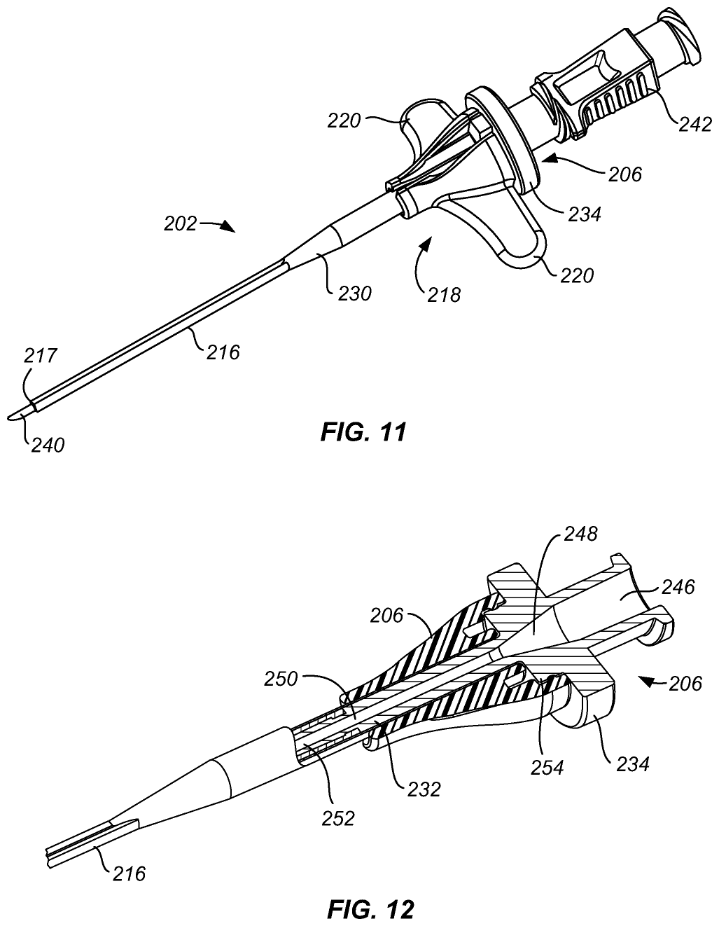

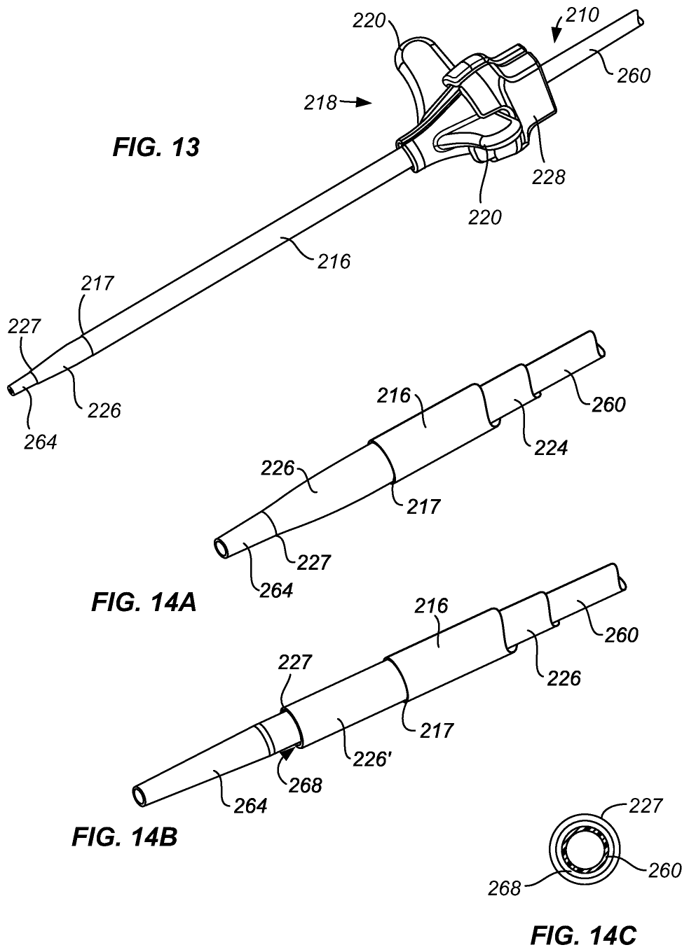

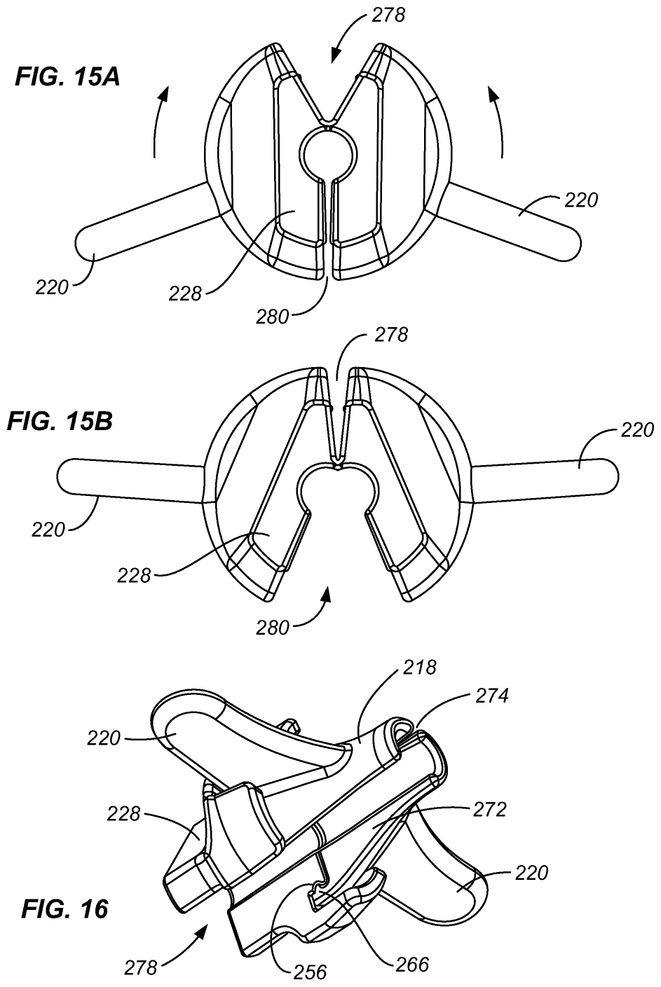

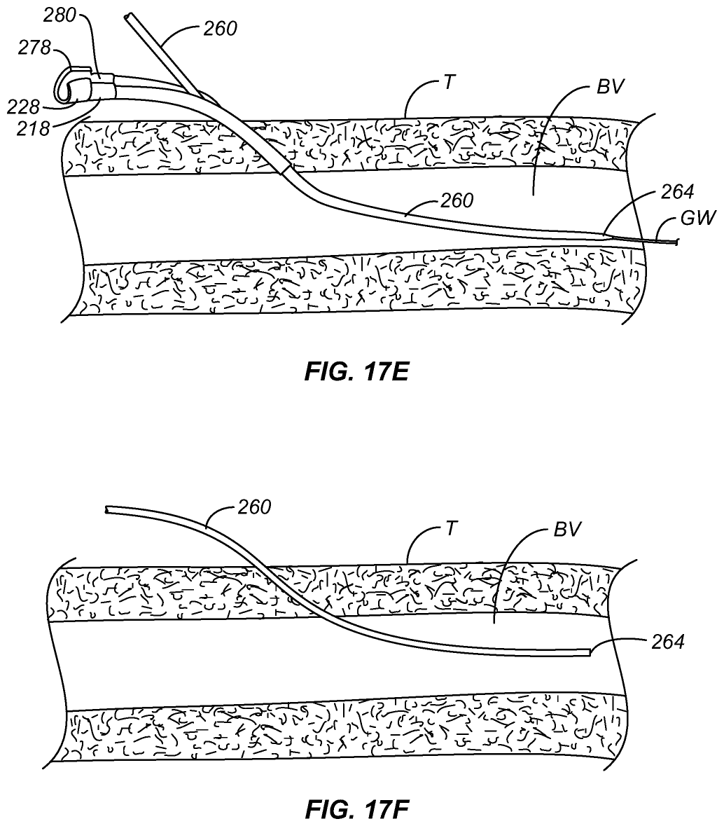

Referring now to FIGS. 13 and 14A to 14C, the radially expandable sheath 202 is radially expanded by advancing the carrier dilator 204 (which has been preloaded with a distal portion of the vascular catheter 210) through a lumen of the tubular sheath body 216. The carrier dilator 204 comprises A tubular carrier body 224 of the carrier dilator has a tapered distal end 226, a distal port 227, and a proximal hub 228. The tubular carrier body 224 of the carrier is split or splittable along its axial length to that it will separate as the carrier dilator 204 is pulled proximally and removed over the catheter body, as shown in FIG. 17E described hereinafter. In most instances, the tubular carrier body 224 will be pre-split along a single axial line (not shown in the drawings) that aligns with the break 280 in the proximal carrier hub 228. As shown in FIG. 10, there is tapered transition region 230 between the radially constricted region of the body and the hub prior to radial expansion. The transition disappears after radial expansion.

A tapered distal end 264 of the vascular catheter 210 is advanced distally through an inner lumen of the tubular body 224 of the carrier dilator 204 to form an assembly which is then advanced through the inner lumen of the tubular sheath body 216, thereby radially expanding the tubular sheath body 216, as shown in FIG. 13. Initially, the tapered distal end 264 of the vascular catheter extends a short distance from the distal port 227 of the tapered distal end 226 of the tubular carrier body 224, as shown in FIG. 14A. By further advancing the tapered distal end 264 of the vascular catheter 210, the tapered distal and 226 of the tubular carrier body 224 is radially expanded to a generally cylindrical configuration 226′, as shown in FIG. 14B. Such expansion will create an annular gap 268, as shown in FIG. 14C, which facilitates free advancement of the vascular catheter within the tubular carrier body 224, allowing the catheter to be fully advanced into the vasculature after initial placement.

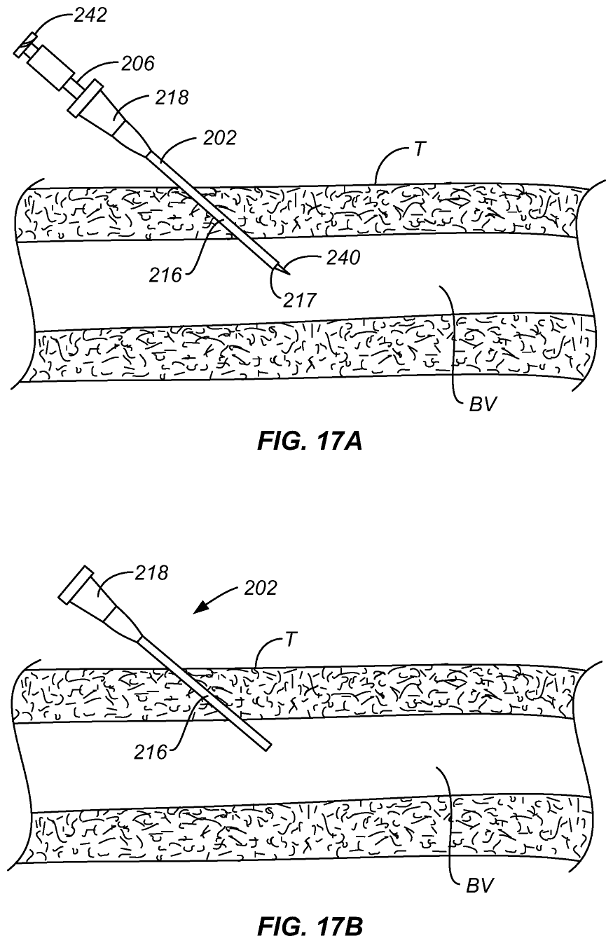

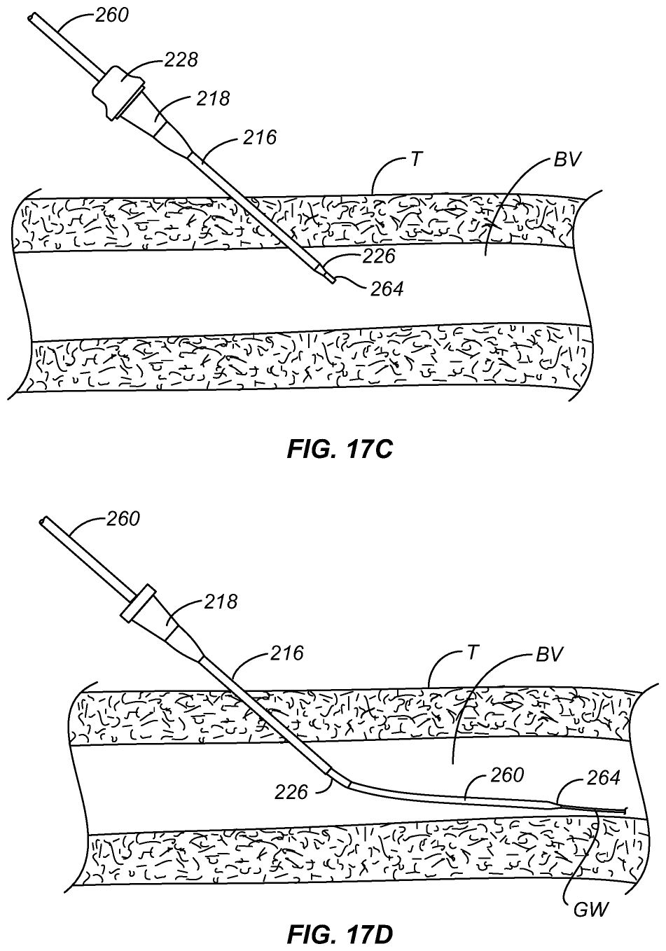

Referring now to FIGS. 15A, 15B, and 16, the proximal hubs 218 and 228 of the radially expandable sheath 202 and the carrier dilator 204, respectively, are usually snapped or otherwise coupled together after the carrier dilator has been fully introduced into the sheath. For example, a thread or detent 266 on an inner surface of the sheath proximal hub 218 can mate with a corresponding groove or thread 256 on an inner surface of the carrier dilator hub 228, as shown in FIG. 16. The proximal sheath hub 218 also has a V-notch 272 formed on one side thereof and a break 274 formed on the diametrically opposite side thereof. Similarly, the proximal carrier dilator hub 2/28 has a V-notch 278 formed on one side thereof and a break 280 formed on the diametrically opposite side thereof. The opposed notches 272 and 278 and breaks 274 and 280 allow the joined hubs 218 and 228 to be simultaneously opened by applying an opening force to the wings 220 in the direction of the arrows in FIG. 15A. While the force is applied directly by the wings only to the proximal sheath hub 218, it is transmitted to the attached carrier dilator hub 228 so that both hubs are opened simultaneously. Opening of the hubs is necessary to allow the radially expandable sheath 202 and the carrier dilator 204 to be removed from over the vascular catheter 210 after the catheter has been properly placed, as described in more detail below.

Referring now to FIGS. 17A to 17F, the percutaneous access system 200 may be used to introduce the vascular catheter 210 to a blood vessel BV as follows. An assembly of the access needle 208, needle guide 206, and radially expandable sheath 202 his first introduced to the blood vessel through the patient's tissue T, as shown in FIG. 17A. The sharpened distal tip 240 of the access needle 208 is advanced through the overlying tissue layer into a lumen of the blood vessel to position the distal end 217 of the radially constricted tubular sheath body 216 therein.

After removing the access needle 208 and the needle guide 206 from the radially constricted tubular sheath body 216, as shown in FIG. 17B, an assembly of the carrier dilator 204 and the vascular catheter 210 is advanced through the proximal hub 218 and tubular body 268 of the radially expandable sheath 202 so that the tubular body is fully expanded and the tapered distal ends 226 and 264 of the tubular carrier body 224 and the catheter 210, respectively, extend into the blood vessel lumen, as shown in FIG. 17C.

The tapered distal end of 264 of the vascular catheter 204 is then advanced distally through the tubular carrier body 224 of the carrier dilator 204, typically over a guidewire GW, as shown in FIG. 17D. Such advancement opens the tapered at distal and 226 of the tubular catheter body 224 to a generally cylindrical configuration as shown at 226′ in FIG. 17D. As the tubular carrier body 224 is non-elastic, and usually at least partially malleable as previously described, the gap 268 (FIGS. 14B and 14C) Will open to reduce contact between an inner surface of the tubular carrier body 224 and the outer surface of the vascular catheter 204, reducing any friction or “stiction” that might otherwise interfere with catheter advancement.

After the vascular catheter 204 has been advanced to its desired position within the lumen of the blood vessel BV, as shown in FIG. 17D, it is necessary to remove both the radially expandable sheath 202 and the carrier dilator 204 from over the catheter body, as shown in FIG. 17E. The hubs 218 and 228 of the radial expandable sheath 202 and the carrier dilator 204 our first opened by applying manual force to the wings 220 of the sheath hub 218, as described previously with reference to FIGS. 15A, 15B and 16. Opening the hubs, in turn, causes the axial split bodies 216 and 224 to also open and allow the catheter body 216 to pass through both the hubs and the tubular bodies which in turn allows both the readily expandable sheath 202 and the carrier dilator 204 to be pulled from the tissue tract, leaving the vascular catheter 210 in place, as shown in FIG. 17F.

While the present invention has been described herein with respect to certain illustrated embodiments, those of ordinary skill in the art will recognize and appreciate that it is not so limited. Rather, many additions, deletions, and modifications to the illustrated embodiments may be made without departing from the scope of the invention as claimed, including legal equivalents thereof. In addition, features from one embodiment may be combined with features of another embodiment while still being encompassed within the scope of the invention as contemplated by the inventors. Further, embodiments of the disclosure have utility with different and various tool types and configurations.

1.-20. (canceled)

21. A percutaneous access system for placement of a vascular catheter through a tissue access tract into a patient's vasculature, said system comprising:
an access needle;
a radially expandable sheath comprising a tubular sheath body having a distal end, a proximal end, and a sheath lumen therebetween, wherein the tubular sheath body is configured to be radially expanded from a radially constricted configuration to a radially expanded configuration and wherein the sheath lumen is configured to receive the needle when said sheath body is in its radially constricted configuration; and
a carrier dilator comprising a tubular carrier body having a distal end, a proximal end, and a carrier lumen therebetween, wherein the carrier lumen is configured to carry the vascular catheter and the distal end the tubular carrier body is tapered to dilate the tubular sheath body as the carrier dilator is advanced through the sheath lumen to expand the sheath body from its radially constricted configuration to its radially expanded configuration.

22. The percutaneous access system of claim 21, wherein the radially expandable sheath further comprises a sheath proximal hub.

23. The percutaneous access system of claim 22, wherein the carrier dilator further comprises a carrier proximal hub configured to detachably couple to the sheath proximal hub.

24. The percutaneous access system of claim 23, wherein the sheath proximal hub and the carrier dilator hub are pre-split or splittable individually and when coupled.

25. The percutaneous access system of claim 23, wherein the tubular sheath body and the tubular carrier body are pre-split or splittable.

26. The percutaneous access system of claim 21, further comprising a needle guide configured to be removably placed in the sheath proximal hub and having a central passage which guides a distal end of the access needle into the sheath lumen.

27. The percutaneous access system of claim 21, further comprising the vascular catheter.

28. The percutaneous access system of claim 27, further comprising a guidewire configured to be received in a guidewire lumen of the vascular catheter and introduced with the catheter into the tubular body of the carrier dilator.

29. The percutaneous access system of claim 21, wherein the radially expandable sheath comprises tubing formed from one or more polymers selected from a group consisting of fluorinated ethylene propylene (FEP), polytetrafluoroethylene (PTFE), ethylene tetrafluoroethylene (ETFE), polyoxymethylene (POM), polybutylene terephthalate (PBT), polyether block ester, polyurethane, polypropylene (PP), polyethylene (PE), low density polyethylene (LDPE), high density polyethylene (HDPE), linear low density polyethylene (LLDPE), polyester, polyamide, elastomeric polyamides, silicones, poly-ethylene terephthalate (PET), polyetheretherketone (PEEK), polyimide (PI), polyetherimide (PEI), polyphenylene sulfide (PPS), polyphenylene oxide (PPO), polysulfone, nylon.

30. The percutaneous access system of claim 29, wherein the radially expandable sheath comprises tubing consists essentially of fluorinated ethylene propylene (FEP).

31. The percutaneous access system of claim 29, wherein the tubing has a wall thickness in a range from 0.01 mm to 0.2.

32. The percutaneous access system of claim 29, wherein the polymer has a density in a range from 2 g/cm³ to 2.2 g/cm³, an elongation-at-break above 200% in a range from 200% to 400%, a flexural modulus in a range from 100 MPa to 1000 MPa, a tensile strength in a range from 0.25 GPa to 1 GPa.

33. A method for placing a vascular catheter through a tissue access tract into a patient's vasculature, said method comprising:
percutaneously inserting a distal end of a tubular sheath body of a radially expandable sheath into a blood vessel in the patient's vasculature, wherein the tubular sheath body is in a radially constricted configuration while it is being inserted; and
advancing a tubular carrier body of a carrier dilator through a lumen of the tubular sheath body which radially expands the tubular sheath body from its radially constricted configuration to a radially expanded configuration, wherein a distal portion of the vascular catheter is disposed in a lumen of the tubular body of the carrier dilator while the tubular carrier body is being introduced.

34. The method of claim 33, further comprising advancing the distal portion of the vascular catheter from the lumen of the tubular body of the carrier dilator into the blood vessel while the distal ends of the tubular sheath body of the radially expandable sheath and the tubular carrier body of a carrier dilator remain in the blood vessel.

35. The method of claim 34, further comprising:
splitting hubs attached to proximal ends of the radially expanded sheath and the carrier dilator, respectively, before or after the distal portion of the vascular catheter has been advanced into the blood vessel; and
pulling the split hubs attached to remove the tubular bodies of the radially expanded sheath and the carrier dilator from over the vascular catheter while the distal portion of the vascular catheter remains in the blood vessel.

36. The method of claim 35, wherein the hubs attached to the proximal ends of the radially expanded sheath and the carrier dilator radially are split and removed simultaneously.

37. The method of claim 36, wherein the sheath proximal hub on the radially expandable sheath and the carrier proximal hub on the carrier dilator are coupled together after the tubular carrier body of the carrier dilator has been advanced through the lumen of the tubular sheath body and before the hubs are split.

38. The method of claim 33, further comprising confirming that the distal portion of the radially expandable sheath has entered the blood vessel prior to advancing the tubular carrier body of the carrier dilator through the lumen of the tubular sheath body.

39. The method of claim 38, wherein confirming comprises ultrasonic imaging.

40. The method of claim 38, wherein confirming comprises observing blood flashback.

41. The method of claim 33, wherein the vascular catheter carries a guidewire in a guidewire lumen thereof when the distal portion of the vascular catheter is carried in the tubular carrier body.

42. The method of claim 41, further comprising advancing the guidewire from the guidewire lumen of the vascular catheter before or after the tubular catheter body has been advanced into the vasculature.

43. The method of claim 42, further comprising advancing the catheter over the guidewire to a target location in the patient's vasculature before or after the guidewire has been advanced.

44. The method of claim 43, wherein the blood vessel is any one of an internal jugular vein, a subclavian vein, an axillary vein, or a femoral vein.

45. The method of claim 33, wherein percutaneously inserting the distal end of the tubular sheath body of the radially expandable sheath into the blood vessel comprises placing an access needle into the lumen of the tubular sheath body so that a sharpened tip of the needle extends distally of the distal end of the tubular sheath body and simultaneously inserting the access needle and the radially expandable sheath.

46. The method of claim 45, wherein advancing the access needle into the lumen of the tubular sheath body comprises attaching a needle guide to a proximal end of the sheath lumen and inserting the access needle through the needle guide.

47. The method of claim 33, further comprising loading a guidewire into a guidewire lumen of the vascular catheter prior to disposing the distal portion of the vascular catheter into the lumen of the tubular body of the carrier dilator.
